SEQUENCE LISTING (10 sequences)

SEQ ID NO: 1 - DNA, 25 bp, synthetic construct
  misc_feature 1..23: note = RNA
  misc_feature 24..25: note = DNA
  source 1..25: mol_type = other DNA
gctcttcccctagataaccaatcat

SEQ ID NO: 2 - RNA, 27 nt, synthetic construct
  source 1..27: mol_type = other RNA
atgattggttatctaggggaagagcag

SEQ ID NO: 3 - DNA, 25 bp, synthetic construct
  misc_feature 1..23: note = RNA
  misc_feature 24..25: note = DNA
  source 1..25: mol_type = other DNA
ggctagatcctcactttctgaacaa

SEQ ID NO: 4 - RNA, 27 nt, synthetic construct
  source 1..27: mol_type = other RNA
ttgttcagaaagtgaggatctagcctg

SEQ ID NO: 5 - DNA, 25 bp, synthetic construct
  misc_feature 1..23: note = RNA
  misc_feature 24..25: note = DNA
  source 1..25: mol_type = other DNA
attgagatcagcgacatcaagtacc

SEQ ID NO: 6 - RNA, 27 nt, synthetic construct
  source 1..27: mol_type = other RNA
ggtacttgatgtcgctgatctcaatgg

SEQ ID NO: 7 - DNA, 4905 bp, Homo sapiens
  source 1..4905: mol_type = genomic DNA
gcagaagtttgcagagcgctttctcgccggctggtcccggactcgcgctgcgccctgcgccctgcgccgccgcctcctccttctgccgccgccgccctgttctcccgggattccttctctgtggaggactgactctctgcttgtgggaattagaggtggacaagggggagtgccggggggtagcgacggggttctttccagtggcagctgcgagtcgggacgccgggagcgtggggctcctgctggccacggatctcggccaccttcccgcggcctccgggcaggcaatgcggccgccgagccccgcgacgcagagaggaaggagggtggcccggagggcctagagcttgtttctgcccacttgggaaagagggcatggagttgtgagtgcccctcgctcggcgacgccgccctcggcaggctccccatggccgggacgtacagctcgactctgaagacgctggaggacttgaccttggactccgggtatggggccggggactcgtgccgctcgctcagcctctcgtcctccaagtccaactcgcaggcgctcaactcttcggcgcagcagcaccgcggggcggcctggtggtgctactccggctccatgaacagccgccacaacagctgggacacggtgaacacggtgctgcccgaggaccccgaagtggccgacctcttctcgcgctgtccgcggctccccgagctggaggagttcccctggaccgaaggagacgtggcccgggtgctccgcaaaggcgctggcggccggcggctgccccagttctccgccgaggcggtgaggcgcctggccgggctgctccgcagggcactgatccgcgtggcccgcgaggcgcagcgcctgagcgtgctgcacgccaagtgcacccgctttgaggtgcagagcgccgtgcgcctggtgcacagctgggcgctggccgagagctgcgcgctggcagccgtcaaggcgctgtccctgtacagcatgagcgccggcgacgggctgcgccggggcaagtccgcgcgctgcggcctcaccttctcagtgggtcgctttttccgctggatggtggacacccgaatctccgtgcgcatccacgagtacgcagccatctccctcaccgcctgcatggagaacctggtggaggagatccgggccagggtgatggccagccacagccctgatggcggaggggccggaggcggggaggtgtctgctgaggccctggagatggtcatcaacaacgacgccgagctctggggcgtcttgcagccctatgagcatctcatctgcggcaagaacgccaatggtgtcctctccctccccgcatacttcagcccctacaacggcgggtccctgggccatgacgagcgagccgatgcctatgcccagctggagctccgaaccctggagcagtccctcctggccacctgcgtgggcagcatctcggagctgagtgacttggtctcccgtgccatgcaccacatgcaggggcgtcaccccctgtgcccgggtgccagccctgcccgccaggcccgccagccgccacagcccatcacttggtcccccgacgccctccacacgctctactactttctgcggtgtccacagatggagtccatggagaaccccaacctggaccccccgagaatgaccttgaacaatgaacggcccttcatgctgctgccgcccctcatggagtggatgcgcgtggccatcacctacgcagagcaccgccgcagcctcaccgtggacagcggcgacatccggcaggcagcccggctgctgctgcctggtctggactgtgaacctcggcagctcaaacccgaacactgtttcagttccttccggaggctggatgcccgagcagctactgaaaaattcaaccaggacctgggtttccgcatgctcaactgtgggaggacggacctcatcaaccaagccatcgaggccttgggtccggatggggttaacaccatggatgaccagggtatgacgccactgatgtacgcctgcgctgctggggacgaagcgatggtccagatgttgattgatgctggggcaaacttggacatccaggttccaagcaactcccccaggcacccttccatccaccccgacagccggcactggacctcactgacattcgctgtgctgcatggacacatctctgtggtccagttgctgctggatgctggtgcccatgtcgagggctcggcagtgaacggcggcgaggacagctatgcggagacgcccctgcagctggcctctgcggcagggaactatgagctggtcagtttgttgctgagccgaggcgccgaccccctcctcagcatgctggaggcccacggcatgggctcctccctccacgaggacatgaactgcttcagccactcagctgcccacggccacaggaacgtcctgaggaagctcctgacgcagccacagcaggctaaagcggacgtgctgtccctggaggagatcctggccgagggtgtggaggaaagtgatgcgtcgagccagggcagtggcagcgaggggcccgtgcggctgagccggacccgcaccaaggccctacaggaggccatgtactacagcgctgagcacggctacgtggacatcaccatggagctgagggcactgggagtcccctggaagctgcacatctggatcgagtctctgaggacctcgttctcgcagtcgcggtactcggtggtgcagagcctcctgcgggacttcagctccatcagagaggaggagtacaacgaggagctggtgaccgagggcttgcagctcatgttcgacatcctcaagaccagcaaaaacgactccgtcatccagcaactggctaccatcttcacccactgctatggcagcagtcccatccccagcatcccagagatccggaagaccctgccggccaggctagatccacactttttgaacaataaggagatgtcagatgtgaccttcctggtggaaggaaagctgttttatgcacataaagtcctgctggtgacagcttctaacaggttcaagacactaatgaccaataaatcagaacaggatggggacagcagcaagaccatcgagatcagcgacatgaagtaccacatttttcagatgatgatgcagtatctgtactacggaggaacagaatccatggagatccccaccactgacatcctggagctgctgtcagctgccagcctgttccagctggatgccctgcagaggcactgcgagatcctgtgctcccagaccctcagcatggagagtgccgtgaacacctacaaatatgccaagatccacaatgccccagaactggccctgttctgtgagggcttcttcctcaagcacatgaaggccctactggagcaggatgccttccggcagctcatctacggccgcagcagcaaagtgcagggcctggatccactgcaggacctgcagaacaccctggcagagcgcgtgcactctgtctacatcacctcccgggtgtgaggcagggggcggaggctgccgaggccagggcctgtggaggtgccagggcccaccatgtcggggtatggttgtaggccctcttggagccaggttcacggggctgtttcctcctcctcccccagatgtcccccctgtcaccgggcagctcaccctcatctcctctgccctagagctgtttgttcaatcactgatgggcaaaggggagatgtagctaactggtggccctcctccccatctcagtttgcaactcaagggcagccatgcactcccccggggtgtcgctctcccctcctgcaggtcagcagctgatcctgatccacctccgcccaggccctccaagctctactcacccctagggcctcagtaaactcagaccatcccaactggggctcctgggggtacaggaatccttatgaatgtatagaatgcatactgcctaggcccccagtcgtctgcagtaattcaaaagctggtatttttcttcctaaatgtcacatagcaaaggctgcccaaatccccaaacttgttcttcaaaggtttgacaggcttaatgtgggcatccagtggcctgtggacccccccctgccaccacgactgctgccacccggagttcccccattgtctttggtgcctgggcttccagagtccctcccatcaggactaccagcttgctcagttagcagcagagggcagttccccagcttgatggtttctgagctaagtctggcatggaggggcaaggggtaggggatggggtgctgtgtaggaatggcctcagctcttgttttgctgctggttttaggggccaaagtaaatgggtggattctggaacctcaggcctagactaggttgcctttgtgggggcgatgaagcagagtcccctggtcaagtctctcggctcctttaatgtaggattgagagctgggcaaacaggtcccactggggactgtgactcttggagattaaaggacagggcattgatggaaggtaagggccaggagaagaggcctttctgctgtgtcccctcacggccttgccctcgaattctgcaggccctgggctcccctgacacctgtcatatttatggaccagacactggggcagcatcaggcacagtctgtctgggtgcaacttcttcagctgtctggccgcaccttaaaaactatttattcaccaaaagccccaggcatttcagaaggtggcactcaggtcaccctgaaaagcccccaggggcttgcaattctggaaaatattttgactagctgcttggctggatgtacaaagaaataggcattattttattgctgtaatattgtatgatactttaactgtacaataatgttgtcacttactgtaaatgtactatggttttattaacaataaacactcattaacaatga

SEQ ID NO: 8 - protein, 1025 aa, Homo sapiens
MAGTYSSTLKTLEDLTLDSGYGAGDSCRSLSLSSSKSNSQALNSSAQQHRGAAWWCYSGSMNSRHNSWDTVNTVLPEDPEVADLFSRCPRLPELEEFPWTEGDVARVLRKGAGGRRLPQFSAEAVRRLAGLLRRALIRVAREAQRLSVLHAKCTRFEVQSAVRLVHSWALAESCALAAVKALSLYSMSAGDGLRRGKSARCGLTFSVGRFFRWMVDTRISVRIHEYAAISLTACMENLVEEIRARVMASHSPDGGGAGGGEVSAEALEMVINNDAELWGVLQPYEHLICGKNANGVLSLPAYFSPYNGGSLGHDERADAYAQLELRTLEQSLLATCVGSISELSDLVSRAMHHMQGRHPLCPGASPARQARQPPQPITWSPDALHTLYYFLRCPQMESMENPNLDPPRMTLNNERPFMLLPPLMEWMRVAITYAEHRRSLTVDSGDIRQAARLLLPGLDCEPRQLKPEHCFSSFRRLDARAATEKFNQDLGFRMLNCGRTDLINQAIEALGPDGVNTMDDQGMTPLMYACAAGDEAMVQMLIDAGANLDIQVPSNSPRHPSIHPDSRHWTSLTFAVLHGHISVVQLLLDAGAHVEGSAVNGGEDSYAETPLQLASAAGNYELVSLLLSRGADPLLSMLEAHGMGSSLHEDMNCFSHSAAHGHRNVLRKLLTQPQQAKADVLSLEEILAEGVEESDASSQGSGSEGPVRLSRTRTKALQEAMYYSAEHGYVDITMELRALGVPWKLHIWIESLRTSFSQSRYSVVQSLLRDFSSIREEEYNEELVTEGLQLMFDILKTSKNDSVIQQLATIFTHCYGSSPIPSIPEIRKTLPARLDPHFLNNKEMSDVTFLVEGKLFYAHKVLLVTASNRFKTLMTNKSEQDGDSSKTIEISDMKYHIFQMMMQYLYYGGTESMEIPTTDILELLSAASLFQLDALQRHCEILCSQTLSMESAVNTYKYAKIHNAPELALFCEGFFLKHMKALLEQDAFRQLIYGRSSKVQGLDPLQDLQNTLAERVHSVYITSRV

SEQ ID NO: 9 - DNA, 4595 bp, Mus musculus
  source 1..4595: mol_type = genomic DNA
gtagaagtttgtggagcgctttctcagcggctggtcccagacggacctcgggcgcctcgctcttcggtggccgccccgttttctcgggattctttctgtggtgatggactctccggacgctctgtaagggaagctgagagctggcggcgcgagttcagaggaggagggctgggaagtccgttcgctgacagcggcgcgactgaacgcggacggcgtcgggctccggccggctgagagcttcgcctaccttcccgcggcctgtgcgcgggcgatgctgcggccccgaggcgcggcgcggcgagcacggagggcgcctccgagagcctagagctggtttccgcccccttcccccggggcaagtgggcatggaacagtgagagcccctcgctcggcgaggccgcgcgcggaggctcccccatggccgggacatacagctccacattgaagactctggaggacttgaccttggactccgggtatggggccggcgactcctgccgctcgctcagcctttcgtcttccaagtccaactcgcaggcgctcaactcttcggcgcagcagcaccgcggggcggcctggtggtgctactccggctccatgaacagccgccacaacagctgggacacggtgaacacagtgctgcccgaggatcccgaagtggccgacctcttctcccgctgcccgcggctcccggagctggaggagttcccttggaccgagggagacgtggcccgggtgctccgcaaaagcgtcggcggccggaggctgccctcgttctccgccgaggctgtgagacgcttggccggactcctccgccgggcgctcatccgcgtggcccgggaggcgcagcgccttagcgtgctgcacgctaagtgcacccgcttcgaggtgcagagtgcggtgcgcctggtgcacagctgggcgctggccgagagctgcgcactggcagcggtcaaggcgttgtccctctacagcatgagcgctggggatgggctgcgccgcggcaagtcggcgcgctgcggcctcactttctccgtgggccgcttcttccgttggatggtggacactcgcatctccgtgcgcatccatgagtacgcggccatctcgctaacggcttgcatggagaacctggtggaggagatccgggccagggtgctggcgagccagagcccagatggcggaggggcaggaggtggagaggtatccgctgaggccttggagatggtcatcaacaacgacgccgagctctggggagtcctgcaaccctatgagcatctcatctgtggcaagaacgccaatggtgtcctctctctccctgcctacttcagtccctacaatggtgggtccctgggccacgatgagcgagcagatgcctatgctcagctggaacttcggacactggagcagtcactcctggccacgtgtgtgggcagcatctccgagctcagtgatctggtctctcgagccatgcaccacatgcagggccgccaccccctgtgcccaggcaccagccctgctcgccaggcccgccagccaccacagcccatcacgtggtcccccgatgctctccacacactctactacttcctgaggtgtccacagatggagtccatggagaaccccaacctggatcccccaagaatgactctgaataatgaacggcccttcatgctgctgccaccgctgatggagtggatgcgggttgcaatcacctatgcagaacaccgtcgcagccccactgtggacagtggcgatatccggcaggcagcccggctactgctgccaggcctggactgtgagccccggcaactcaaacccgagtgctgcttcagttccttccggaggctggatgctcgagctgctactgaacgcttcaaccaggacctgggcttccgcatgctcaactgtgggagaacagacctcatcagccaggccatcgaggccctgggcccggatggggtgaacacaatggatgaccagggtatgacgccgcttatgtacgcctgtgctgctggggacgaagccatggtccagatgctgattgatgctggagctaacttagatatccaggttccaagccactctcccaggcatccgtccgtccaccccgacagcaggcattggacctccctgacatttgcggtgctacatggacacatctctgtggtgcagctgctacttgatgccggtgcccatgtggagggctcagctgtgaacagtggggaggacagctacgccgagacacccctgcagctggcctctgcagccgggaactatgagctggtcagcttgctgctgagccggggtgctgaccccctcctcagcatgctggaagccaacggcatggcatcttccctccacgaggagatgaactgcttcagccactccgctgcccacggccacagaaatgtcctgagaaagctcctgactcagccacagcaggctaaagcagatgtgctgtctctagaagagatcctggctgagggtgtggaggaaagcgacacatcgagccagggcagcagtgaggggcctgtgaggctaagccgaacccgaacgaaggccctgcaggaagccatgtactacagcgctgagcacggttacctggacatcaccatggagctgagggcactgggtgttccctggaagctgcacatctggatagagtcgctgcggacctccttctcacagtcacggtactcagtggtgcagagcctcctgaaggatttcagctccatcaaggaagaggagtacaacgaggagctggtgacagaaggcttgcagctcatgtttgatatcctcaagaccagcaaaaacgactccgtcctccagcagctggccaccatcttcacacactgctatggcaccagccccatccccagcatcccggagatccggaagaccctgccagccaggctagatcctcactttctgaacaacaaggagatgtctgatgtaaccttcctagtggaaggaaagctgttttatgcacacaaagtccttttggtgacagcatctaacaggttcaagaccttaatgaccaacaaatcagaacaagatggggacagcagcaaaaccattgagatcagcgacatcaagtaccacatctttcagatgctgatgcagtacctgtactacggaggaaccgagtccatggaaatccccactgctgacatcctgcagctgctgtcagctgccaacctgttccagctagatgccctgcagagacactgtgagatcctgtgctcccagacccttagtgtggaaagtgctgtcaacacctacaaatatgccaagatccacaacgccccggagctggccctcttctgtgaaggcttcttcctcaagcacatgaaggccctgctagagcaggatgccttccggcagctcatctatggccgaagcagcaaagtccagggcctggacccactgcaggatctgcagagcaccctagcagagcgagtgcattctgtctacgtcacctcccgggtgtgaggcaggatggagggggactgttatctgagggcgtgcaaaggtctgttaaggccagaaaccaccagtgacacccccagtggaaggaggcttgtaggctttaggggtacttcctgctcttcccctagataaccaatcatctgtggtgttgaccgacccttgggtgaacagagtccacttcactgttgctcaaccagtaactggcaagggggtgtaacatagttgcaccttgagggccaccctgtgccctctcccctccagtggctcagcagcaagctcacctcagctccagctctcgccatcctggctggactgttaggggcatgggcattcttatgaatgtagaaaatggatcctgagccccaggccactggatgcagtagctggtatgctttgttttcctaaacagagaagaaagggtttccaaatcctgtcctccaagctgtgcctttgaaagggctggtgaggtggcccctgtctgtgaagactggacctctagtcgcctgccccacgagactactatgttaggagtctgagctcagagcttccgagctgatgatgggctacagtacaggaactcaaggacttccgctcttgctgctacaactggcagccaaagtgaagaggttggttctaggtcgggcctagctctaatgtgtctgcaggctgaggcagcagagtgccttccagttaggacaggggtgcaagagcacacttgtcacctgggatctgtgactctggggcatgaaggacaaggcctcggtggaagagaagctatgtcccctcccatcctgtcctcagtcttggctccccatcctctggcctgtttgtggacagatgctgggacagcatcaagaacagctaccttagaaactatttattcatcagaagcccccagggcttgcaatcctggaaagatattttcaacttgctgcttgtgcgggatgtacaaaggaatgaatggtattttattgctgtaatattgtataatactgtaactataccttaatgttgttacttactgtaaatgtgctatggttttatcaacaataaacactcattaacattgaaaaaaaaaaaaaaaaaaaaaaaaaaaaaaaaaaaaa

SEQ ID NO: 10 - protein, 1024 aa, Mus musculus
MAGTYSSTLKTLEDLTLDSGYGAGDSCRSLSLSSSKSNSQALNSSAQQHRGAAWWCYSGSMNSRHNSWDTVNTVLPEDPEVADLFSRCPRLPELEEFPWTEGDVARVLRKSVGGRRLPSFSAEAVRRLAGLLRRALIRVAREAQRLSVLHAKCTRFEVQSAVRLVHSWALAESCALAAVKALSLYSMSAGDGLRRGKSARCGLTFSVGRFFRWMVDTRISVRIHEYAAISLTACMENLVEEIRARVLASQSPDGGGAGGGEVSAEALEMVINNDAELWGVLQPYEHLICGKNANGVLSLPAYFSPYNGGSLGHDERADAYAQLELRTLEQSLLATCVGSISELSDLVSRAMHHMQGRHPLCPGTSPARQARQPPQPITWSPDALHTLYYFLRCPQMESMENPNLDPPRMTLNNERPFMLLPPLMEWMRVAITYAEHRRSPTVDSGDIRQAARLLLPGLDCEPRQLKPECCFSSFRRLDARAATERFNQDLGFRMLNCGRTDLISQAIEALGPDGVNTMDDQGMTPLMYACAAGDEAMVQMLIDAGANLDIQVPSHSPRHPSVHPDSRHWTSLTFAVLHGHISVVQLLLDAGAHVEGSAVNSGEDSYAETPLQLASAAGNYELVSLLLSRGADPLLSMLEANGMASSLHEEMNCFSHSAAHGHRNVLRKLLTQPQQAKADVLSLEEILAEGVEESDTSSQGSSEGPVRLSRTRTKALQEAMYYSAEHGYLDITMELRALGVPWKLHIWIESLRTSFSQSRYSVVQSLLKDFSSIKEEEYNEELVTEGLQLMFDILKTSKNDSVLQQLATIFTHCYGTSPIPSIPEIRKTLPARLDPHFLNNKEMSDVTFLVEGKLFYAHKVLLVTASNRFKTLMTNKSEQDGDSSKTIEISDIKYHIFQMLMQYLYYGGTESMEIPTADILQLLSAANLFQLDALQRHCEILCSQTLSVESAVNTYKYAKIHNAPELALFCEGFFLKHMKALLEQDAFRQLIYGRSSKVQGLDPLQDLQSTLAERVHSVYVTSRV

(19) United States
(12) Patent Application Publication
(10) Pub. No.: US 2023/0310659 A1
(43) Pub. Date: Oct. 5, 2023

(54) ANKYRIN-RICH BTB/POZ DOMAIN-CONTAINING PROTEIN-2 (ABTB2) FOR SUPPRESSING PANCREATIC CANCER GROWTH

(71) Applicant: The Curators of the University of Missouri, Columbia, MO (US)

(72) Inventors: Guangfu LI, Columbia, MO (US); Kevin STAVELEY-O'CARROLL, Columbia, MO (US)

(21) Appl. No.: 18/194,691

(22) Filed: Apr. 3, 2023

Related U.S. Application Data
(60) Provisional application No. 63/326,415, filed on Apr. 1, 2022.

(51) Int. Cl. A61K 48/00; A61P 35/00; C07K 14/47; C12N 15/86

(52) U.S. Cl. CPC A61K 48/0066; A61P 35/00; C07K 14/47; A61K 48/0041; C12N 15/86; C12N 2750/14143

(57) ABSTRACT

Disclosed are compositions and methods of treating cancer. The compositions include an Ankyrin-rich BTB/POZ domain-containing protein-2 (ABTB2)-expressing virus and are particularly useful for treating pancreatic cancer.

### CROSS-REFERENCE TO RELATED APPLICATIONS

This application claims priority to U.S. Provisional Patent Application No. 63/326,415, filed on Apr. 1, 2022, which is incorporated by reference in its entirety.

### INCORPORATION OF SEQUENCE LISTING

A computer readable form of the Sequence Listing containing the file named “3513285.0402 Sequence Listing.xml”, which is 23,511 bytes in size (as measured in MICROSOFT WINDOWS® EXPLORER) and was generated on Mar. 30, 2023, is provided herein and is herein incorporated by reference. This Sequence Listing consists of SEQ ID NOs:1-10.

### BACKGROUND OF THE DISCLOSURE

The present disclosure relates generally to medicine. More particularly, the present disclosure is directed to compositions and methods for treating cancer. The compositions and methods are particularly suitable for treating pancreatic cancer.

Pancreatic cancer (PaC) is a highly lethal malignancy in humans and the fourth leading cause of cancer-related death. Conventional therapies, including surgery, chemotherapy, and radiation therapy, provide only limited benefit to patients. Surgery, as a potential cure, is available to very few patients, as more than 80% of patients are inoperable when diagnosed with PaC. PaC is also unusually resistant to all forms of cytotoxic chemotherapy.

Molecularly targeted therapy (MTT) is a new generation of cancer treatment approved by the Food and Drug Administration (FDA) to treat many types of cancer. MTT targets specific molecules involved in tumor growth and spreading. However, ongoing and completed clinical trials of MTTs that focus on previously identified genes have not been effective in human patients with PaC, as the median overall survival of patients is still less than one year.

Accordingly, there exists a continuing need for developing new compositions and methods for treating cancer and, particularly, for treating pancreatic cancer.

### BRIEF DESCRIPTION OF THE DISCLOSURE

The present disclosure relates generally to compositions and methods for treating cancer. The compositions and methods are particularly useful for treating pancreatic cancer.

In one aspect, the present disclosure is directed to vector comprising a nucleic acid encoding an Ankyrin-rich BTB/POZ domain-containing protein-2 (ABTB2).

In one aspect, the present disclosure is directed to a method for treating cancer in a subject in need thereof, the method comprising: administering to the subject a composition comprises a vector comprising a nucleic acid encoding an Ankyrin-rich BTB/POZ domain-containing protein-2 (ABTB2).

### DESCRIPTION OF THE DRAWINGS

FIG. 1 depicts decreased ABTB2 protein production in PaC tumors compared to peri-tumor tissue with anti-ABTB2 Ab (PAS-54135, ThermoFisher) by immunohistochemistry.

FIGS. 2A-2F depict the establishment of stable Panc02 cells with ABTB2 knockout and overexpression. FIGS. 2A and 2D show schematics of establishing stable cell lines for ABTB2-knockout with CRISPR and ABTB2 ectopic expression with recombinant plasmid. FIGS. 2B and 2E show mRNA expression detected by qPCR in the indicated stable cells. FIGS. 2C and 2F show ABTB2 protein expression detected by western blotting in the indicated stable cells.

FIGS. 3A-3N depict the impact of Abtb2 defect on PaC progression in vitro and in vivo. Results indicated that ABTB2 overexpression (OE) reduced cell proliferation (FIG. 3A), decreased colony formation (FIG. 3C), increased cell-free gap (FIGS. 3E and 3F), and therapeutically reduced tumor growth (FIGS. 31 and 3J). The opposite results were observed in stable ABTB2 knockout (KO) cells (FIGS. 3B, 3D, 3G, 3H, 3K, and 3L). Significantly extended and shortened survival times were detected in mice receiving stable ABTB2 OE cells (FIG. 3M) and ABTB2 KO cells (FIG. 3N). n=10, *P<0.05, ** P<0.01, *** P<0.001.

FIGS. 4A-4D depict therapeutic suppression of mABTB2-AAV2 PaC tumor growth. Panc02 cells were seeded into wild type C57BL/6 mice by intra-pancreatic injection at a dose of 0.25×10⁶ cells/mouse. On day 11, all mice were randomly divided into three groups and received i.v. injection of mABTB2-AAV2 virus at 1×10¹¹ VG/mouse. PBS and AAV2 vector virus were used as two controls (FIG. 4A). On day 35, the tumor size in the treated mice was smaller than that in two control groups of mice (FIGS. 4B and 4C). Significantly extended survival times were detected in the treated mice relevant to two control mice (FIG. 4D). n=10, *P<0.05.

FIGS. 5A and 5B depict tumor growth as monitored with MRI. C57BL/6 mice received an intrapancreatic inoculation of Panc02-H7 cells at a dose of 0.25×10⁶ cells/mouse on day 0, then underwent MRI on Day 5 (FIG. 5A) and Day 15 (FIG. 5B). Tumors are outlined.

FIG. 6 is a photograph of an exemplary PDX mouse model created by subcutaneously inoculating an immunodeficient NSG mouse with 3 ˜1 mm³ pieces of freshly harvested tumors from human patients. Arrow points to the formed tumors on day 40 after tumor tissue transplantation.

FIG. 7 is a graph depicting the design and identification of siRNA that specifically suppressed abtb2 gene expression in PaC cells. siRNA-1, siRNA-2, and siRNA-3 significantly suppressed abtb2 mRNA expression to different extents with about 35% reduction observed for siRNA-1 and siRNA-2.

FIG. 8 is a graph demonstrating that siRNA-2 significantly suppressed abtb2 mRNA expression in Panc02 cells. The results indicate that siRNA-2 significantly suppressed abtb2 mRNA expression in Panco2 cells to 20% level in normal cells (NC) without siRNA transfection or transfected with scramble siRNAs (SC).

FIGS. 9A-9C depict the preparation of stable mouse PC cell line overexpressing Abtb2. FIG. 9A illustrates transfection of HEK-293 cells with the Abtb2 recombinant plasmids and two other vectors to prepare Abtb2-recombinant lentivirus, which was then used to transfect Panc02 cells. FIG. 9B depicts the Abtb2 overexpression in cell clones B10, F9, and D9 as detected by qPCR. FIG. 9C depicts the Abtb2 protein expression in clone B10 cells relative to that in vehicle control cells as detected by Western blot.

FIGS. 10A-10C depict the development of stable Abtb2-knockout cell line using CRISPR. FIG. 10A depicts the preparation of stable Abtb2 knockout in Panc02 cells with CRISPR technology. FIG. 10B depicts CRISPR-mediated knockout of Abtb2 in cell clones 2, 3, 4, 5 and 9 with almost no detectable Abtb2 in clone 4. FIG. 10C depicts a Western blot showing no observable ABTB2 protein was detected in stable Abtb2-CRISPR-Panc02 cells relative to vehicle control cells.

FIGS. 11A and 11B depict the impact of ABTB2 knockdown on Panc02 cell colony formation. Representative images (FIG. 11A) and accumulated results (FIG. 11B) show increased cell colonies in Abtb2-siRNA-transfected Panc02 cells.

FIGS. 12A and 12B depict the impact of ABTB2 overexpression on Panc02 cell colony formation. Representative images (FIG. 12A) and accumulated results (FIG. 12B) show the reduced cell colonies in Abtb2-overexpressed stable cells.

FIGS. 13A and 13B depict suppression of Panc02 cell motility by ABTB2 overexpression. FIG. 13A depicts representative images for the cell-free gap in the indicated cells and FIG. 13B depicts the accumulated results of cell-free gaps in the different cells. n=3, error bars represent mean±SD.

FIGS. 14A and 14B depict the impact of ABTB2 knockdown on Panc-1 cell colony formation. Representative images (FIG. 14A) and accumulated results (FIG. 14B) show increased cell colonies in Abtb2-siRNA-transfected Panc-1 cells.

FIG. 15 depicts the impact of siRNA-mediated ABTB2 knockdown on Panc-1 cell proliferation.

FIGS. 16A-16C ABTB2 overexpression suppressed orthotopic pancreatic tumor growth in wild type mice. Representative results show tumors and different organs (FIG. 16A). Accumulated results of tumor sizes (FIG. 16B). n=5, error bars represent mean±SD. (C) ABTB2 overexpression did not change the weight of liver, spleen, kidney, and lung (FIG. 16C).

FIG. 17 ABTB2 overexpression induced apoptosis of Panc02 cells by suppressing Bcl-2 and caspase-3 pathways. The decreased expression of Bcl-2 and cleaved caspase-3 and increased expression of Bax and Bcl-XL were detected in Abtb2-overexpression cells.

FIGS. 18A and 18B ABTB2 overexpression suppressed orthotopic pancreatic tumor growth in immunodeficiency mice. Representative results show tumors and different organs (FIG. 18A). Accumulated results of tumor sizes (FIG. 18B). n=5 for ABTB2-overexpression cell-induced tumors, n=4 for control cell-induced tumors. error bars represent mean±SD.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

While the present disclosure is susceptible to various modifications and alternative forms, exemplary embodiments thereof are shown by way of example in the drawings and are herein described in detail. It should be understood, however, that the description of exemplary embodiments is not intended to limit the disclosure to the particular forms disclosed, but on the contrary, the intention is to cover all modifications, equivalents and alternatives falling within the scope of the disclosure as defined by the embodiments above and the claims below. Reference should therefore be made to the embodiments above and claims below for interpreting the scope of the present disclosure.

Unless defined otherwise, all technical and scientific terms used herein have the same meaning as commonly understood by one of skill in the art to which the invention pertains. Although any methods and materials similar to or equivalent to those described herein can be used in the practice or testing of the present disclosure, the preferred methods and materials are described herein. Moreover, reference to an element by the indefinite article “a” or “an” does not exclude the possibility that more than one element is present, unless the context clearly requires that there be one and only one element. The indefinite article “a” or “an” thus usually includes “at least one.” The term “about” means up to ±10%.

As used herein, “susceptible” and “at risk” refer to having little resistance to a certain disease, disorder or condition, including being genetically predisposed, having a family history of, and/or having symptoms of the disease, disorder or condition.

As used herein, a subject in need thereof, as it relates to the therapeutic uses herein, is one identified to require or desire medical intervention. Because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects in need of treatment described herein. An effective amount is that amount of an agent necessary to inhibit the pathological diseases and disorders herein described. When at least one additional therapeutic agent is administered to a subjects, such agents may be administered sequentially, concurrently, or simultaneously, in order to obtain the benefits of the agents. The term subject includes vertebrate animals, and preferably is a human patient.

In one aspect, the present disclosure is directed to a vector having a nucleic acid encoding an Ankyrin-rich BTB/POZ domain-containing protein-2 (ABTB2).

Ankyrin-rich BTB/POZ domain-containing protein-2 (ABTB2) contains ankyrin repeat (AR) and BTB/POZ domains, belonging to BPOZ domain-containing proteins. The BTB (for BR-C, ttk and bab)[2] or POZ (for Pox virus and Zinc finger)[3] domain is present near the N-terminus of a fraction of zinc finger proteins and in proteins that contain the Kelch motif and a family of pox virus proteins. An exemplary embodiment of Homo sapiens ABTB2 is provided in Gene ID:25841. An exemplary embodiment of Mus musculus ABTB2 (also known as BPOZ-2) is provided in Gene ID:99382.

Suitable vectors are viral vectors. Suitable viral vectors include adeno-associated-viral vectors from AAV serotypes such as, for example, adeno-associated-virus serotype-1 (AVV-1), adeno-associated-virus serotype 2 (AAV2), adeno-associated-virus serotype-5 (AVV-5), adeno-associated-virus serotype-6 (AVV-6), adeno-associated-virus serotype-8 (AVV-8), adeno-associated-virus serotype-9 (AVV-9), adeno-associated-virus serotype-rh74 (AVV-rh74), adeno-associated-virus-2i8 (AVV-2i8), adeno-associated-virus-B1 (AVV-B1), adeno-associated-virus-CAM130 (AVV-CAM130), adeno-associated-virus-M41 (AVV-M41), adeno-associated-virus MTP (AAV587MTP and AAV588MTP), adeno-associated-virus NP22 (AAV-NP22), adeno-associated-virus NP66 (AAV-NP66), adeno-associated-virus MYO (AAVMYO), adeno-associated-virus tyrosine mutants, and ancestral adeno-associated-virus (ancAVV).

The nucleic acid encoding an Ankyrin-rich BTB/POZ domain-containing protein-2 (ABTB2) can further include a promotor to drive expression of the nucleic acid. Suitable promoters include a Cox-2 promoter, a P48 promoter, a Pdx1 promoter, a Hnf6 promoter, a ngn3 promoter, a MAFA promoter, a NeuroD/BETA2 promoter, a Pax6 promoter, and a Pax4 promoter. A particularly suitable promoter is a Cox-2 promoter.

In one aspect, the present disclosure is directed to a composition comprising a vector having a nucleic acid encoding an Ankyrin-rich BTB/POZ domain-containing protein-2 (ABTB2).

In one embodiment, the composition is a pharmaceutical composition.

The composition can further include other components such as surfactants, preservatives, and excipients. Suitable surfactants fatty acid esters and alcohols, and polyoxyethylene sorbitol fatty acid esters. Amounts will generally range from about 0.001 and about 4% by weight of the formulation. Pharmaceutically acceptable preservatives include, for example, phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) and mixtures thereof. The preservative can be present in concentrations ranging from about 0.1 mg/ml to about 20 mg/ml, including from about 0.1 mg/ml to about 10 mg/ml. The use of a preservative in pharmaceutical compositions is well-known to those skilled in the art. For convenience reference is made to Remington: The Science and Practice of Pharmacy, 19th edition, 1995. Formulations can include suitable buffers such as sodium acetate, glycylglycine, HEPES (4-(2-hydroxyethyl)-1-piperazineethanesulfonic acid) and sodium phosphate. Excipients include components for tonicity adjustment, antioxidants, and stabilizers as commonly used in the preparation of pharmaceutical formulations. Other inactive ingredients include, for example, L-histidine, L-histidine monohydrochloride monohydrate, sorbitol, polysorbate 80, sodium citrate, sodium chloride, and EDTA di sodium.

The composition can further include a pharmaceutically acceptable carrier. As understood by those skilled in the art, pharmaceutically acceptable carriers, and, optionally, other therapeutic and/or prophylactic ingredients must be “acceptable” in the sense of being compatible with the other ingredients of the formulation and not be harmful to the recipient thereof. Suitable pharmaceutically acceptable carrier solutions include water, saline, isotonic saline, phosphate buffered saline, Ringer's lactate, and the like. The compositions of the present disclosure can be administered to animals, preferably to mammals, and in particular to humans as therapeutics per se, as mixtures with one another or in the form of pharmaceutical preparations, and which as active constituent contains an effective dose of the active agent, in addition to customary pharmaceutically innocuous excipients and additives.

In one aspect, the present disclosure is directed to a method of treating a subject having or suspected of having cancer, the method comprising: administering to the subject a composition having a viral vector encoding an Ankyrin-rich BTB/POZ domain-containing protein-2 (ABTB2).

Suitably, the viral vector is contained in a virus. Suitable viruses include AAV serotypes such as, for example, adeno-associated-virus serotype-1 (AVV-1), adeno-associated-virus serotype 2 (AAV2), adeno-associated-virus serotype-5 (AVV-5), adeno-associated-virus serotype-6 (AVV-6), adeno-associated-virus serotype-8 (AVV-8), adeno-associated-virus serotype-9 (AVV-9), adeno-associated-virus serotype-rh74 (AVV-rh74), adeno-associated-virus-2i8 (AVV-2i8), adeno-associated-virus-B 1 (AVV-B1), adeno-associated-virus-CAM130 (AVV-CAM130), adeno-associated-virus-M41 (AVV-M41), adeno-associated-virus MTP (AAV587MTP and AAV588MTP), adeno-associated-virus NP22 (AAV-NP22), adeno-associated-virus NP66 (AAV-NP66), adeno-associated-virus MYO (AAVMYO), adeno-associated-virus tyrosine mutants, and ancestral adeno-associated-virus (ancAVV).

The nucleic acid encoding an Ankyrin-rich BTB/POZ domain-containing protein-2 (ABTB2) can further include a promotor to drive expression of the nucleic acid. Suitable promoters include a Cox-2 promoter, a P48 promoter, a Pdx1 promoter, a Hnf6 promoter, a ngn3 promoter, a MAFA promoter, a NeuroD/BETA2 promoter, a Pax6 promoter, and a Pax4 promoter. A particularly suitable promoter is a Cox-2 promoter.

In one embodiment, the subject has or is suspected of having pancreatic cancer.

Suitable methods for administration of formulations of the present disclosure are by parenteral (e.g., intravenous (IV), intramuscular (IM), subcutaneous (SC), or intraperitoneal (IP)) routes and the formulations administered ordinarily include effective amounts of product in combination with acceptable diluents, carriers and/or adjuvants. Standard diluents such as human serum albumin are contemplated for pharmaceutical compositions of the disclosure, as are standard carriers as described herein. The composition can be administered over the course of days, weeks, months, and years.

Formulations for parenteral administration (e.g. by injection, for example bolus injection or continuous infusion) can be presented in unit dose form in ampoules, pre-filled syringes, small volume infusion or in multi-dose containers with and without an added preservative. The formulations can take such forms as suspensions, solutions, or emulsions in oily or aqueous vehicles, and may contain formulation agents such as suspending, stabilizing and/or dispersing agents.

As used herein, an “effective amount”, a “therapeutically effective amount”, a “prophylactically effective amount” and a “diagnostically effective amount” is the amount of the combination therapy of the present disclosure needed to elicit the desired biological response following administration. Suitable dosage for use in the methods of the present disclosure will depend upon a number of factors including, for example, age and weight of an individual, severity of the hepatocellular cancer, nature of a composition, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result.

As used herein, “treating” (or “treat” or “treatment”) refers to processes involving a slowing, interrupting, arresting, controlling, stopping, reducing, or reversing the progression or severity of an existing symptom, disorder, condition, or disease, but does not necessarily involve a total elimination of all disease-related symptoms, conditions, or disorders associated with administration of the therapy.

In some embodiments, treatment results in a decrease or reduction in tumor growth.

Tumor growth can be determined using methods known in the art such as magnetic resonance imaging (MRI), for example.

Protein expression can be determined using methods known in the art such as immunohistochemistry, Western blot analysis, Enzyme Linked Immunosorbent Assay (ELISA), Northern blot analysis, in situ hybridization, and amplification (e.g., polymerase chain reaction including RT-PCR, qPCR, etc.), for example.

### EXAMPLES

Embodiments of the present disclosure are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1

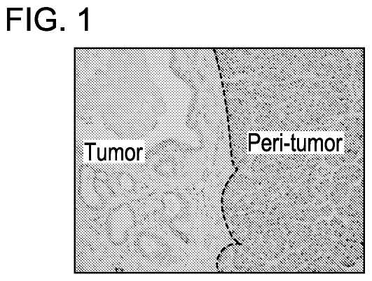

The ABTB2 protein has ankyrin repeat (AR) and BTB/POZ domains, which are found in BPOZ domain-containing proteins. BPOZ proteins participate in protein-protein interactions with their specific ubiquitin ligase and substrates via the BTB domain, mainly to control the degradation of many proteins. In this way, BPOZ proteins regulate a wide range of cellular processes, such as cell growth, apoptosis, and metabolically defective protein clearance. In 2001, ABTB2 was first reported to suppress ovarian tumor cells via the PTEN signaling pathway. In 2004, one group used serial analysis of gene expression (SAGE) to identify ABTB2 among the top 100 overexpressed genes in PaC by measuring their mRNA expression. The Human Protein Atlas database shows that a low expression of ABTB2 is favorable to PaC patient survival at mRNA, but not protein level; intratumoral ABTB2 protein expression is low in some patients and undetectable in most patients; and no patient shows high and medium ABTB2 protein expression. ABTB2 decreased protein expression in human PaC tumors compared to peri-tumoral tissues (FIG. 1). A literature review found no other reports on ABTB2 function in PaC.

Immunohistochemistry (IHC) showed decreased production of ABTB2 protein in human PaC tumors. First, human PaC tumors were harvested to evaluate ABTB2 protein production in PaC. Unlike the increased mRNA expression in PaC tumors shown by SAGE and human Protein Atlas databases, IHC analysis reliably detected less ABTB2 protein in human PaC tumors than in peri-tumor tissue (FIG. 1).

Establishing stable PaC cells with ABTB2 knockout (KO) or overexpression (OE).

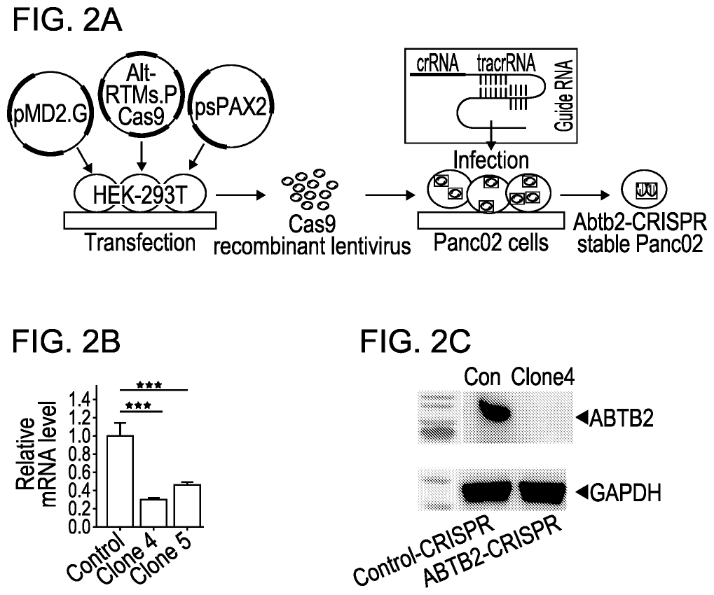

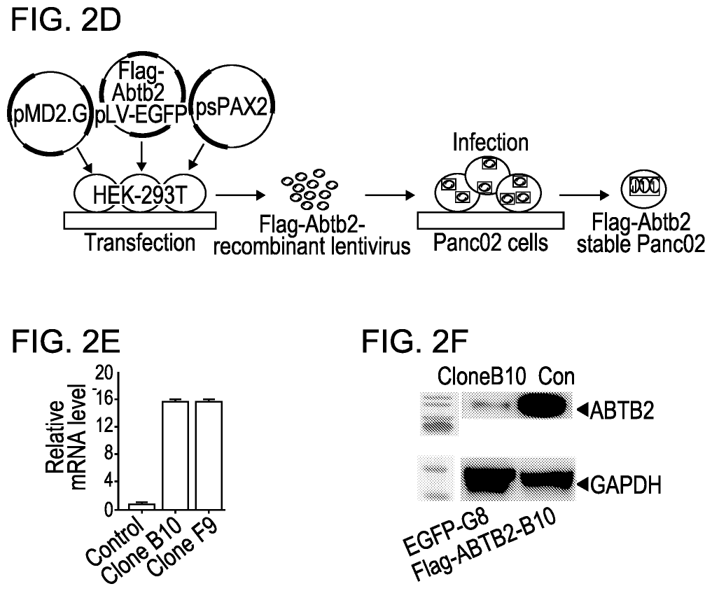

To investigate the role of ABTB2 in PaC progression, stable Panc02 cell lines were established with ABTB2-KO using CRISPR-Cas9 technology (FIGS. 2A-2C) and ABTB2-OE using ABTB2 recombinant plasmid (FIGS. 2D-2F). ABTB2-KO or ABTB2-OE mRNA and protein levels were measured in each cell clone with qPCR and western blot, respectively (FIGS. 2B, 2C, 2E, and 2F). Cell clones with successful ABTB2 KO or OE were respectively named ABTB2-KO and ABTB2-OE. These cells are useful to investigate how intrinsic ABTB2-KO or ABTB2-OE impacts PaC.

Intrinsic ABTB2-KO or ABTB2-OE significantly impacted PaC cell growth in vitro and in vivo.

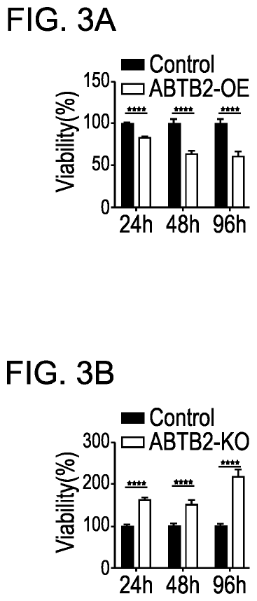

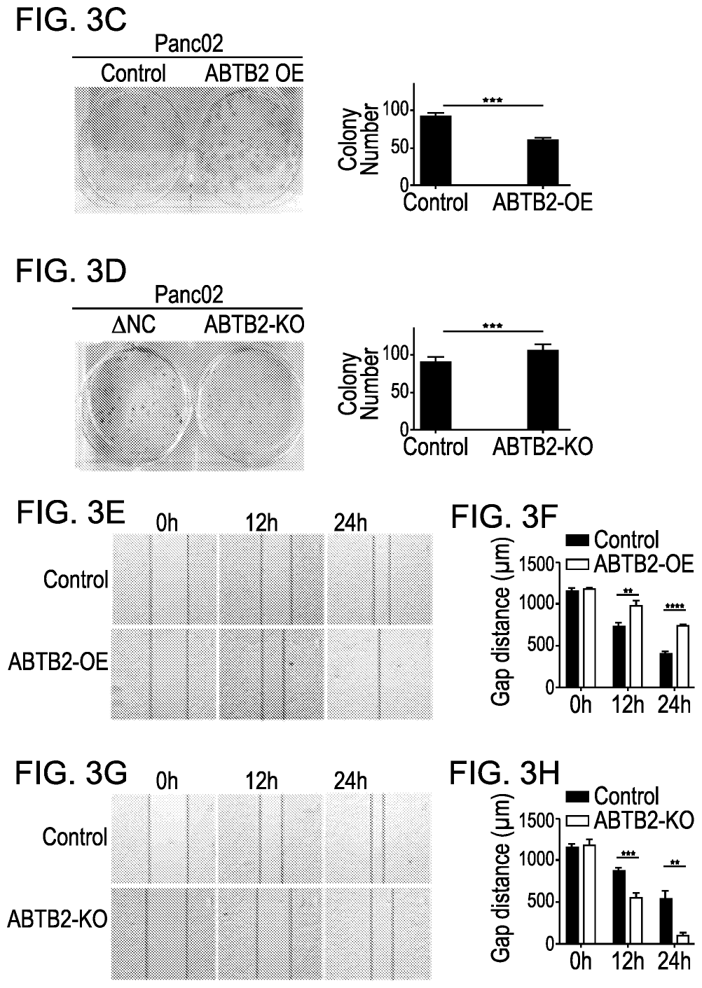

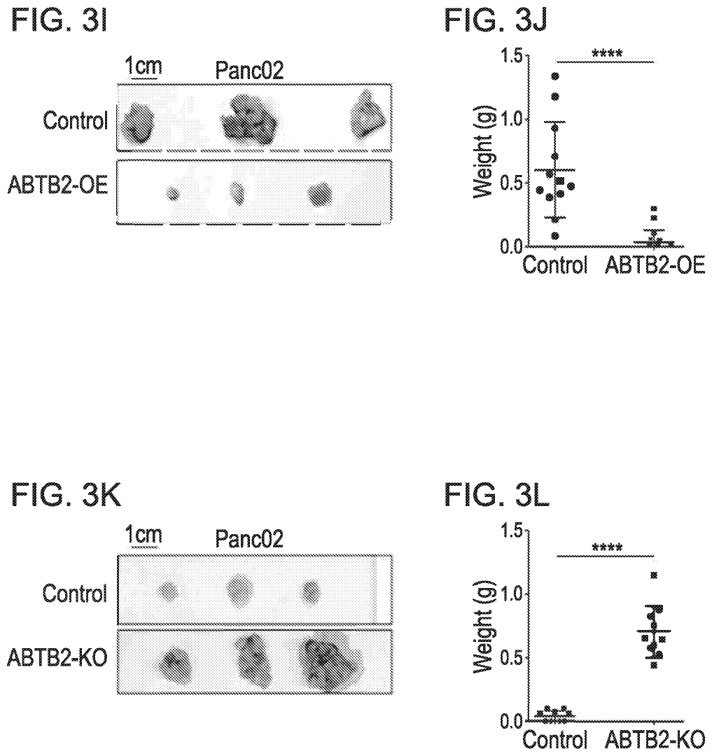

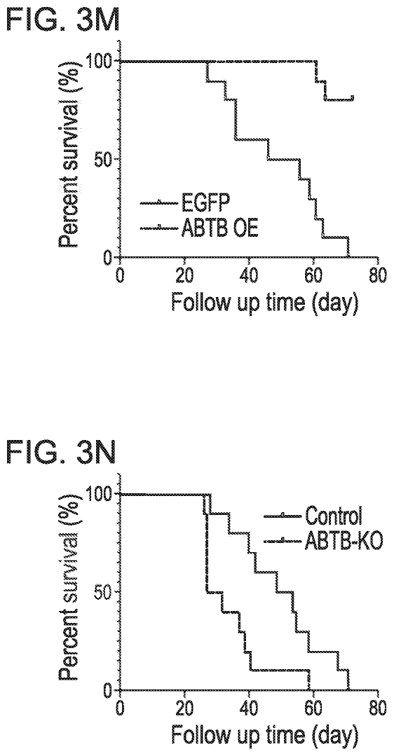

Establishing stable ABTB2-KO cells and ABTB2-OE cells allowed further investigation of how loss or gain of ABTB2 function impacted PaC in vitro and in vivo. Stable ABTB2-KO cells and ABTB2-OE cells were seeded into the plates and wild type C57BL/6 mice by orthotopic inoculation. Overexpressed ABTB2 significantly reduced cell proliferation (p<0.001) (FIG. 3A), colony formation (p<0.001) (FIG. 3C), cell motility (FIGS. 3E and 3F), and tumor growth (p<0.001) (FIGS. 3I and 3J). In contrast, the opposite effects were observed in ABTB2-KO cells. Moreover, ABTB2's suppressive effect on PaC cells was validated in other PaC cells, including mouse UN-KPC-961 cells with mutated Kras (G12D) and Trp53 (R172H), and human Panc-1 and MIA PaCa-2 cells (data not shown). These results indicated that ABTB2 is an unrecognized gene that can suppress PaC.

A new targeted ABTB2 MTT markedly suppressed PaC tumor growth without detectable adverse effect.

Following identification of a pivotal role for ABTB2 in PaC growth and metastasis, an ABTB2-based MTT was designed for PaC treatment. Adeno virus-associated virus serotype 2 (AAV2) was used to construct a mouse ABTB2 recombinant virus, named mABTB2-AAV2, to express mouse ABTB2 under control of a PaC-specific Cox-2 promoter to ensure that mABTB2-AAV2 specifically expressed ABTB2 in PaC tumors in vivo.

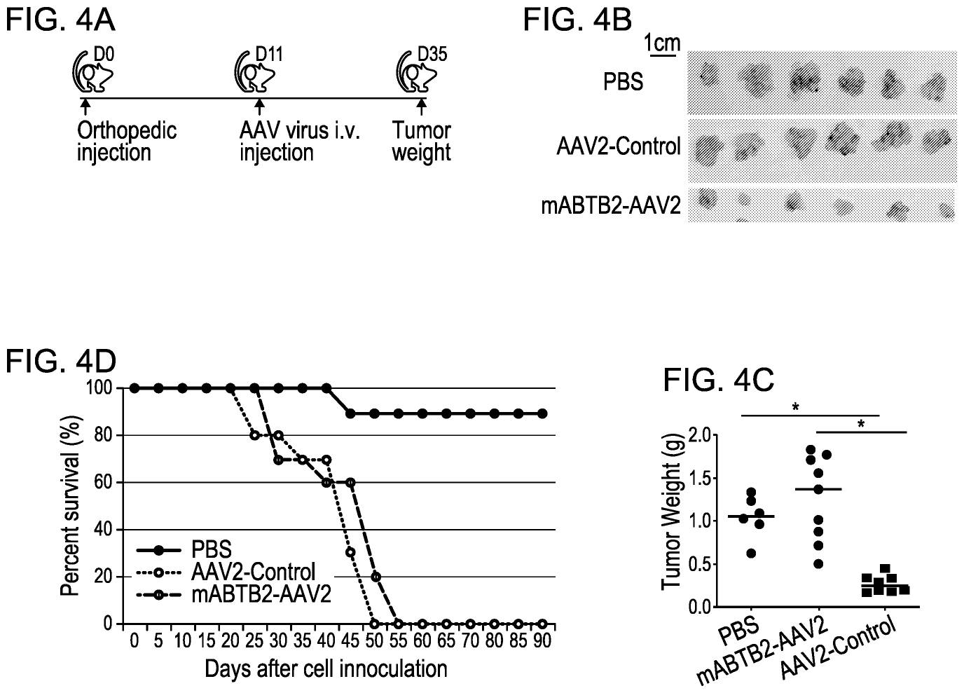

To test the therapeutic effect of mABTB2-AAV2 virus on PaC, orthotopic PaC tumor-bearing mice were made with Panc02 cells as illustrated in FIG. 3. On day 11 after cell inoculation, mice were treated with mABTB2-AAV2 by i.v. injection (FIG. 4A). On day 35, some mice were euthanized to monitor tumor growth. Results indicate that mABTB2-AAV2 treatment suppressed tumor progression (FIGS. 4B and 4C). The remaining mice were maintained to measure lifetime survival. A significantly extended lifetime was detected in mABTB2-AAV2-treated mice compared to the control treatment, and there was no detectable liver, renal, hematologic, or cardiac toxicity (data not shown), as determined by analysis described in Li et al. (Gastroenterology. 2018; 154(4):1024-36 e9. doi: 10.1053/j.gastro.2017.10.050. PubMed PMID: 29408569; PMCID: PMC5908238). Similar therapeutic efficacy was validated in mice with orthotopic PaC tumors induced with UN-KPC-961 cells (data not shown). I.v. injection of mABTB2-AAV2 virus was also observed to cause PaC tumor-specific ABTB2 expression (data not shown). These results indicated that mABTB2-AAV2 virus therapeutically suppressed PaC.

To address an unmet clinical need in PaC MTT, a therapeutic virus was constructed to infect PaC cells and manipulate ABTB2 expression. The therapeutic virus significantly suppressed PaC growth without detectable adverse effects.

In vitro and in vivo studies with different cells and mouse models demonstrated the critical role of ABTB2 in suppressing PaC. A literature review showed no reports for ABTB2's anti-PaC function.

Statistical Considerations. All in vitro experiments were repeated three times and data will be presented as mean±SE. Experiments with >2 levels will be compared using ANOVA, enhancing power. The sample sizes presented were based on achieving ≥90% power to detect ≥1.3-fold differences among groups after the potential loss of 10-15% of mice to attrition. For all experiments, α≤0.05 was considered significant. Transformations (e.g. log) was used as appropriate to meet the assumptions of the statistical tests employed. To achieve the above, experimental groups included 12 mice per group (10 to attain 90% power+2 for attrition).

Example 1 evaluated the impact of ABTB2-targeted therapy on PaC progression, stroma reprogramming, immune reaction, and adverse effects in mouse KPC and human PDX models.

ABTB2 strongly suppressed PaC growth (FIG. 3) and mABTB2-AAV2 virus therapeutically suppressed orthotopic PaC tumors in two murine transplantation models induced with Panc02 or UN-KPC-961 cells (FIG. 4). Testing of ABTB2-AAV2 therapy was expanded to genetically engineered KPC mice and human patient-derived xenografts (PDX) mice. KPC is the most extensively used PaC model, with many features in common with human disease. In this model, tumors arise spontaneously in the pancreas due to Kras and P53 mutation. PDX models have realistic human PaC tumors that retain genetic and biological characteristics of the original human tumors and intact human tumor stromal components, thus more reliably and faithfully predict effects of treatments in cancer patients.

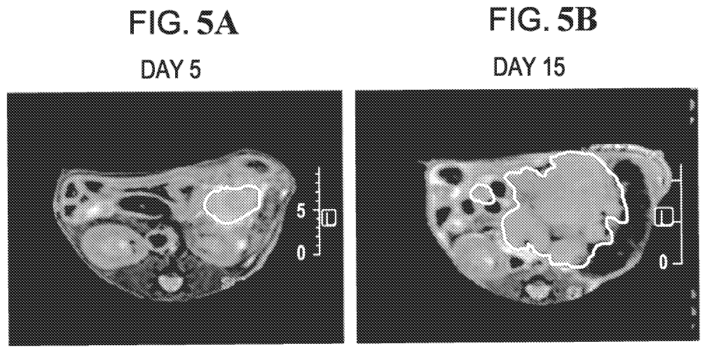

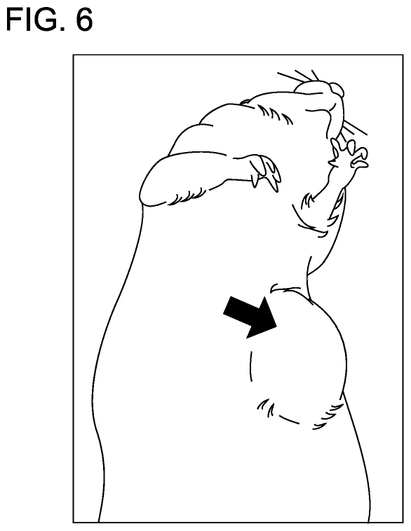

KPC model: The pancreas of newly born KPC mice is normal and devoid of neoplastic cells. However, by 8 to 10 weeks old, KPC mice harbor precursor lesions, or pancreatic intraepithelial neoplasia (PanIN), within the pancreas. By 16 weeks old, most KPC mice have developed locally invasive PaC accompanied by a dense desmoplastic reaction. MRI was used to monitor PaC tumor growth (FIG. 5). PDX model: immunodeficient NSG mice (Stock No: 005557) were obtained from Jackson Laboratory. Subcutaneously inoculating three tumor pieces (1 mm³) freshly harvested from human PaC patients without any treatment (IRB Project No: 2010166), formed and grew tumors (FIG. 6). In addition, some xenografts can be reliably passaged to other mice, enabling the retention of identical tumor-bearing mice for future studies (data not shown).

Examining the therapeutic efficacy of mABTB2-AAV2 in KPC mice to evaluate the impact of ABTB2-targeted therapy on PaC progression, stroma reprogramming, immune reaction, and adverse effects. KPC mice at 8 and 16 weeks old, representing tumor initiation and progression, respectively, were monitored by MRI. Sex- and tumor-size-matched KPC mice were divided into three groups. G1: As depicted in FIG. 4A, mice were orthotopically injected with Panc02 cells at day 0, followed by injection of AAV virus (or controls) at day 11, and determination of tumor weight at day 35.

Panc02 cells were seeded into wild type C57BL/6 mice by intra-pancreatic injection at a dose of 0.25×10⁶ cells/mouse. On day 11, all mice were randomly divided into three groups and received i.v. injection of mABTB2-AAV2 virus at 1×10¹¹ VG/mouse. PBS and AAV2 vector virus were used as two controls. On day 35, the tumor size in the treated mice was smaller than that in two control groups of mice. Results indicate that mABTB2-AAV2 treatment suppressed tumor progression (FIGS. 4B and 4C). The remaining mice were maintained to measure lifetime survival. A significantly extended lifetime was detected in mABTB2-AAV2-treated mice compared to the control treatment, and there was no detectable liver, renal, hematologic, or cardiac toxicity (data not shown). Similar therapeutic efficacy was validated in mice with orthotopic PaC tumors induced with UN-KPC-961 cells (data not shown). I.V. injection of mABTB2-AAV2 virus also caused PaC tumor-specific ABTB2 expression (data not shown). These compelling results suggest that mABTB2-AAV2 virus therapeutically suppresses PaC. Significantly extended survival times were detected in the treated mice relevant to two control mice (FIG. 4D). n=10, *P<0.05.

To monitor tumor growth with Mill, C57BL/6 mice received an intrapancreatic inoculation of Panc02-H7 cells at a dose of 0.25×10⁶ cells/mouse on day 0, then underwent Mill on Day 5 and Day 15 (FIG. 5).

To establish a PDX mouse model, immunodeficient NSG mice received subcutaneous inoculation of 3-˜1 mm³ pieces of freshly harvested tumors from human patients. Arrow points to the formed tumors on day 40 after tumor tissue transplantation.

To examine the therapeutic efficacy of human ABTB2-AAV2 in the PDX model, a human ABTB2-AAV (hABTB2-AAV2) virus was constructed and evaluated for its therapeutic efficacy in human PaC tumors in the first generation of PDX mice. Experimental design: Using the strategy described in FIG. 6, PDX mice were generated using freshly harvested human PaC tumors. In addition, recombinant AAV2 that expresses human ABTB2 was constructed, as depicted in FIG. 4. Age-, sex- and tumor size-matched PDX mice were randomly divided into three groups to receive the following treatments: G1: No treatment; G2: AAV2 virus treatment; G3: hABTB2-AAV2 virus treatment. Mice received a single intra-tumoral injection of hABTB2-AAV2 at a 1×10¹¹ VG/mouse dose. Tumor growth and metastasis, mouse survival, tumoricidal effect, intra-tumoral ABTB2 expression, and toxicity were evaluated in mice under the treatments above. The recombinant mABTB2-AAV2 virus therapeutically suppressed orthotopic PaC tumors formed with Panc02 (FIG. 4) and UN-KPC-961 cells.

To identify ABTB2-specific interacting ubiquitin ligases and substrate proteins relevant to PaC, tagged ABTB2 will be transfected into PaC cells and immunoprecipitation (IP) will be used to pull down ABTB2 complexes to identify ABTB2-specific ubiquitin ligases and substrates by mass spectrometry and western blotting.

ABTB2, as a BPOZ protein, is a strong tumor suppressor. BPOZ proteins use AR and BTB/POZ domains to interact with ubiquitin ligase and substrates, forming a protein complex. STRING database and previous studies revealed that BPOZ domain-containing proteins, as substrate adaptors, interact with ubiquitin ligase Cullin 1 or 3 and substrates, directing specific substrate ubiquitylation and subsequent degradation to mediate different biological functions, including arresting tumor growth and suppressing cell proliferation. Therefore, it is possible that ABTB2, a BPOZ protein, acts as an adaptor by interacting with a specific ubiquitin ligase and substrate to form a complex. The complex suppresses PaC by controlling substrate ubiquitylation and degradation. Identifying ABTB2-specific ubiquitin ligases and substrates will lay a foundation to support future mechanistic studies and provide new targets for developing anti-PaC MTTs.

Immunoprecipitation (IP) with anti-FLAG antibodies will be used to pull down ABTB2 complexes and analyzed using using mass spectrometry and western blotting to identify ABTB2-interacting ubiquitin ligases in PaC cells. Experimental design: Using established approaches, the following studies will be performed to achieve several goals. 1) Tagged ABTB2 construct. Recombinant plasmids will be constructed with pCIN4 vector that expresses the FLAG-tagged C-terminal region of ABTB2. The resultant FLAG-tagged ABTB2 will be transfected into Panco2 cells. Western blot will be used to confirm the expression of the transfected proteins with antibodies for ABTB2 and FLAG. IP will be performed with anti-FLAG antibodies to pull down ABTB2 complexes. The pull-down protein complexes will undergo in-solution digest and subsequent mass spectrometry analysis to identify ABTB2-interacting ubiquitin ligases. Results will identify ABTB2-specific ubiquitin ligases. Western blot will be used to confirm the findings.

Anti-FLAG antibodies will be used to pull down ABTB2 complexes in PaC cells to identify candidate ABTB2-specific substrates with mass spectrometry, then validate the findings by loss and gain of ABTB2 function. ABTB2-interacting ubiquitin ligases and substrates will be identified and validated. Given that BPOZ domain-containing proteins act as substrate adaptors that interact with ubiquitin ligase Cullin 1 or 3, Cullin 1, 2, or 3 may be identified as the ABTB2-specific ubiquitin ligase in PaC. Eukaryotic elongation factor 1A1 (eEF1A1) and terminal deoxynucleotidyltransferase (TdT) are known to be two BPOZ-2-specific substrates, and thus, may be identified as ABTB2-specific substrates.

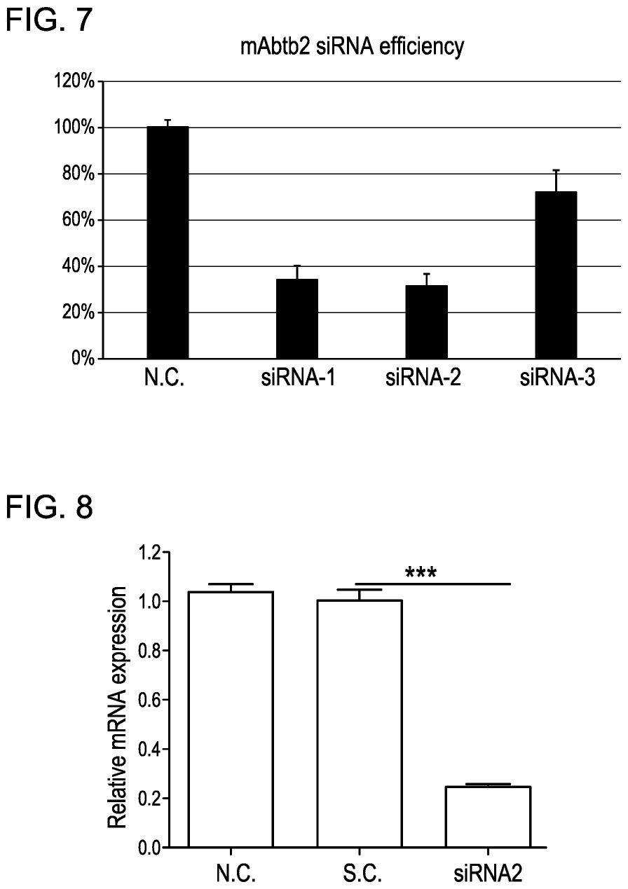

Suppression of abtb2 gene expression in PaC cells using siRNA was analyzed. 1×10⁵ cells were seeded in each well of a 6-well plate and transfected with the indicated siRNAs designed to suppress abtb2 in the second day. Cells were harvested on the third day to extract total RNA to detect abtb2 mRNA expression with real-time PCR. The cells with negative control DsiRNA transfection were used for controls. Results indicated that siRNA-1, siRNA-2, and siRNA-3 significantly suppressed abtb2 mRNA expression to different extents with about 35% reduction observed for siRNA-1 and siRNA-2 (FIG. 7)

To determine if siRNA-2 effectively suppresses abtb2 in Panc02 cells, Panc02 cells were prepared and transfected with siRNA-2 as panc02 cells in FIG. 7. The cells were harvested 24 hours after transfection and used to do real-time PCR. The results indicate that siRNA-2 significantly suppressed abtb2 mRNA expression in Panco2 cells to 20% level in normal cells (NC) without siRNA transfection or transfected with scramble siRNAs (SC) (see, FIG. 8).

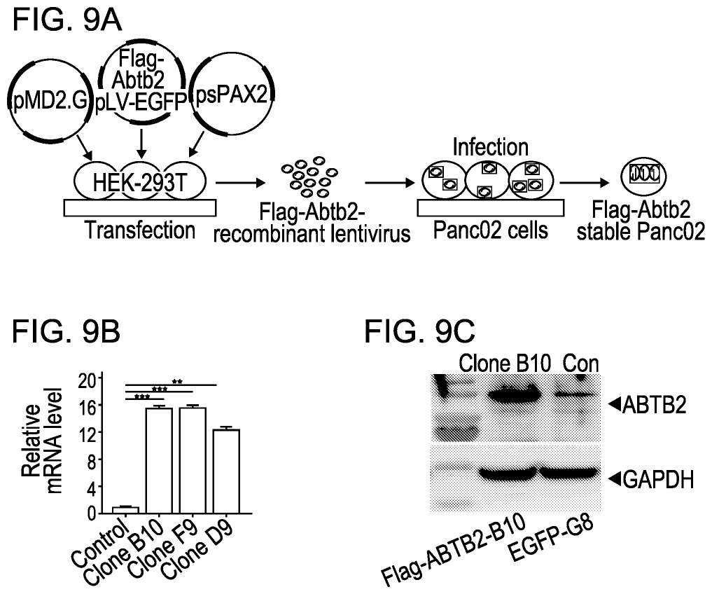

To prepare a stable mouse PC cell line overexpressing Abtb2, HEK-293 cells were transfected with the Abtb2 recombinant plasmids and two other vectors to prepare Abtb2-recombinant lentivirus (FIG. 9A), which was then used to transfect Panc02 cells. On the second day, the cells were diluted and seeded into each well in 96-well plate under antibiotic selection to obtain single cell clones. Abtb2 was overexpressed in cell clones B10, F9, and D9 as detected by qPCR (FIG. 9B). Abtb2 protein expression in clone B10 cells relative to that in vehicle control cells was detected by Western blot (FIG. 9C).

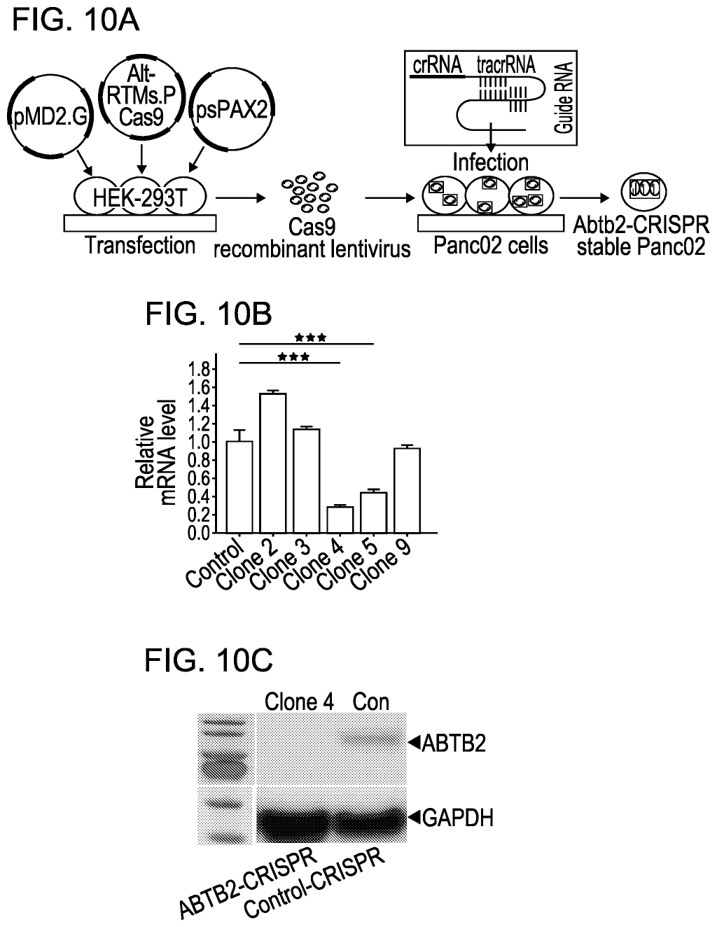

A stable Abtb2-knockout cell line was developed in Panc02 cells using CRISPR technology (FIG. 10A). Following manufacturer's instructions, Cas9 recombinant plasmids were first transfected into Panc02 cells to establish single Cas9-expressing stable cell lines. The designed gRNAs for Abtb2 were transfected into stable Cas9-Panc02 cells with RNAi-MAX Lipofectamine (Invitrogen). The transfected cells were diluted on the second day and seeded into each well to get single cell clones. depicts. Suppressed expression of Abtb2 via CRISPR-mediated knockout of Abtb2 in cell clones 2, 3, 4, 5 and 9 as detected by qPCR with almost no detectable Abtb2 in clone 4 (FIG. 10B). Clone 4-amplified cells were named as Abtb2-CRISPR-Panc02 cells. Western blot showed no observable ABTB2 protein expression detected in stable Abtb2-CRISPR-Panc02 cells relative to vehicle control cells (FIG. 10C).

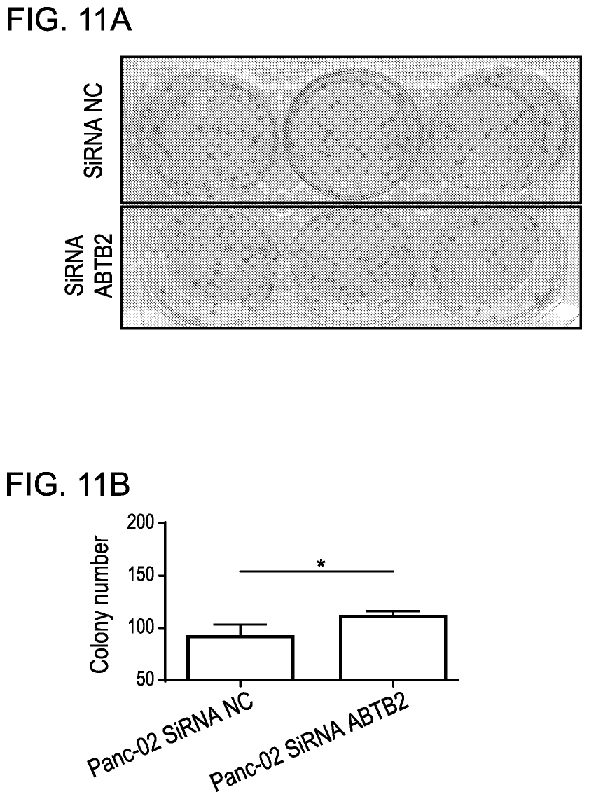

The impact of ABTB2 knockdown on Panc02 cell colony formation showed increased cell colonies in Abtb2-siRNA-transfected Panc02 cells (FIGS. 11A and 11B).

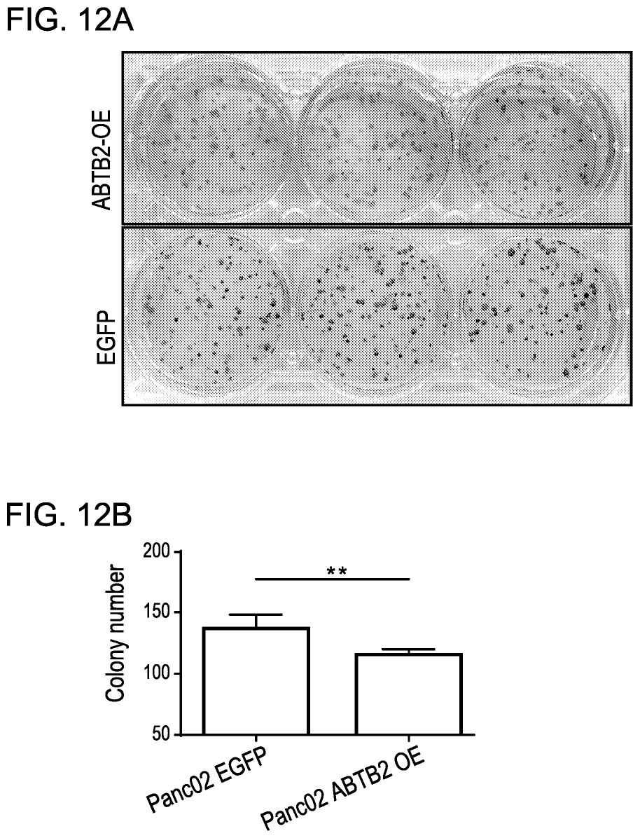

ABTB2 overexpression on Panc02 cell colony formation show reduced cell colonies in Abtb2-overexpressed stable cells (FIGS. 12A and 12B).

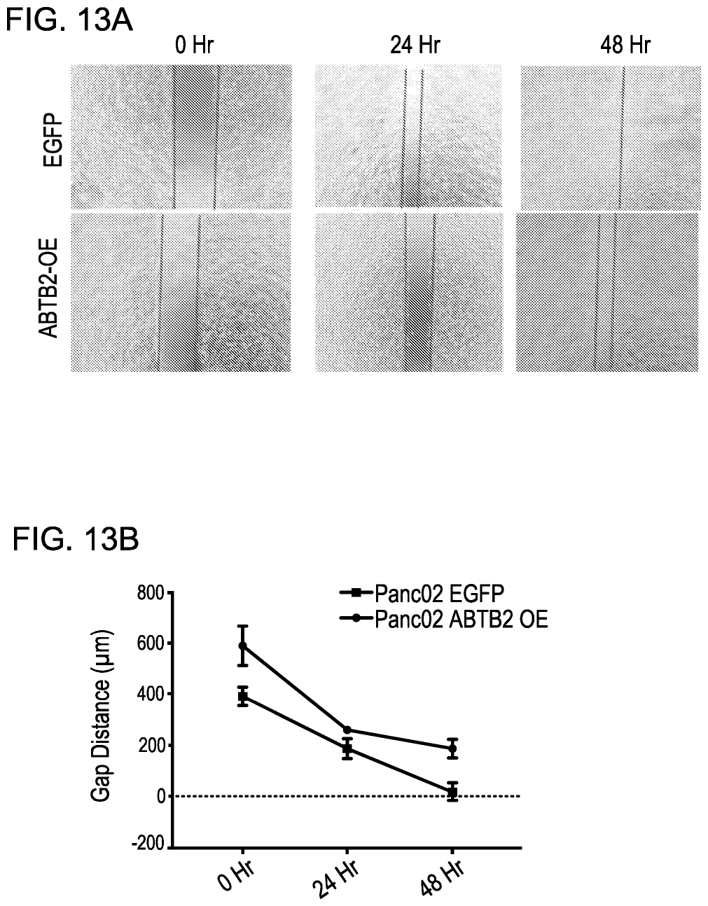

The effect of ABTB2 overexpression on cell motility was determined in Panc02 cells. The ABTB2-overexpression cells and control cells were seeded in 24-well plates with an insert. The next day, the insert was removed. The width of the cell-free gap was measured over the indicated times. Representative images show cell-free gap in the indicated cells (FIG. 13A) and the accumulated results of cell-free gaps in the different cells (FIG. 13B).

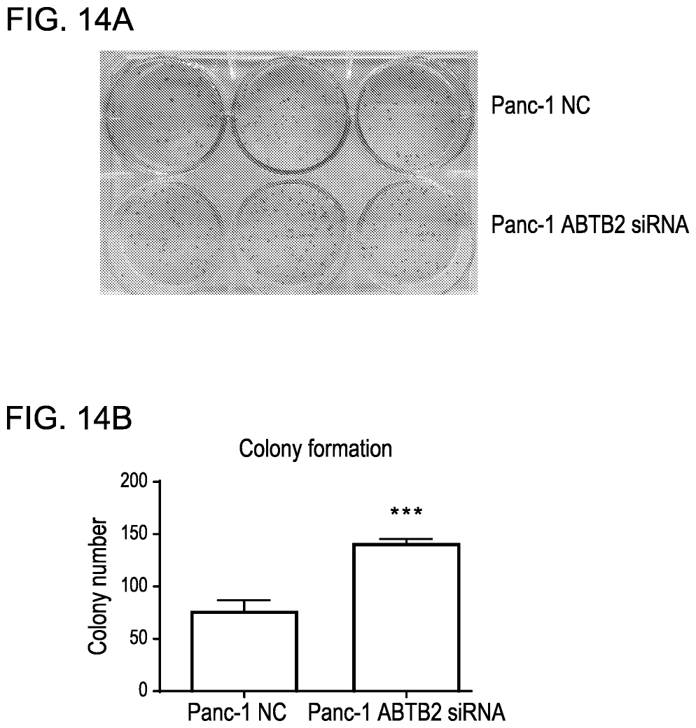

The impact of ABTB2 knockdown on Panc-1 cell colony formation showed increased cell colonies in Abtb2-siRNA-transfected Panc-1 cells (FIGS. 14A and 14B).

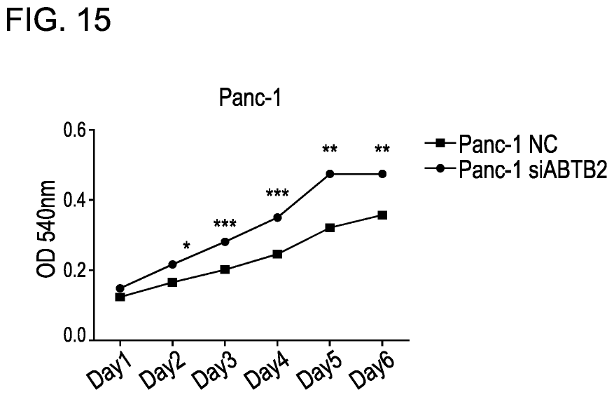

The impact of siRNA-mediated ABTB2 knockdown on Panc-1 cell proliferation was determined. Panc-1 cells were transfected with siRNA. The next day, the cells were harvested and seeded into 96-well plates. Cell proliferation in each well at the indicated times were measured with Proliferation Kit (FIG. 15).

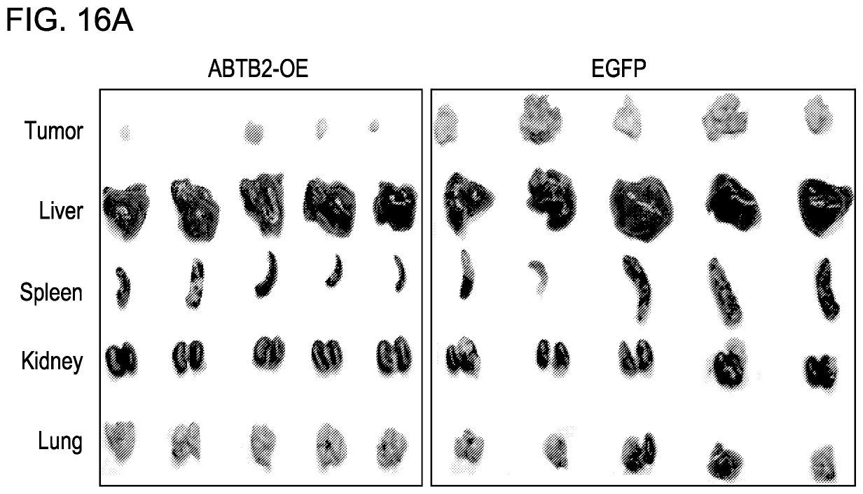

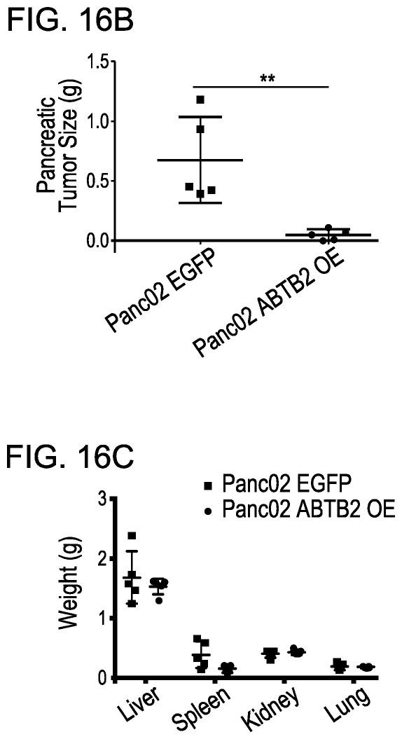

ABTB2 overexpression in orthotopic pancreatic tumor growth in wild type mice was investigated. Stable mouse Panco2 cells with ABTB2-overexpression and control cells were cultured and harvested, then injected into wild type C57BL/6 mice by orthotopic inoculation at a dose of 0.25×10⁶ cells per mouse. 35 Days later, the mice were euthanized to harvest different organs and tumors. The tumors were imaged and weighed. Representative results show tumors and different organs (FIG. 16A). Accumulated results of tumor sizes (FIG. 16B). n=5, error bars represent mean±SD. ABTB2 overexpression did not change the weight of liver, spleen, kidney, and lung (FIG. 16C).

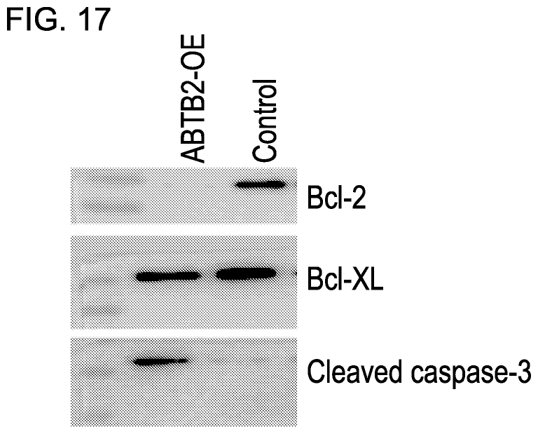

ABTB2 overexpression induced apoptosis of Panc02 cells by suppressing Bcl-2 and caspase-3 pathways. The decreased expression of Bcl-2 and cleaved caspase-3 and increased expression of Bax and Bcl-XL were detected in Abtb2-overexpressing cells (FIG. 17).

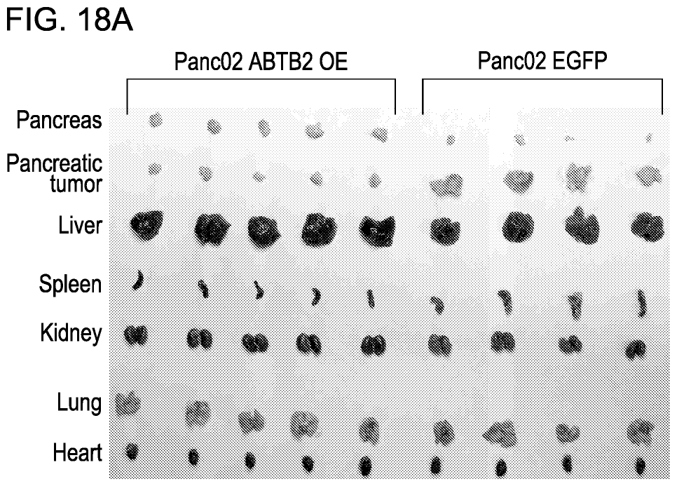

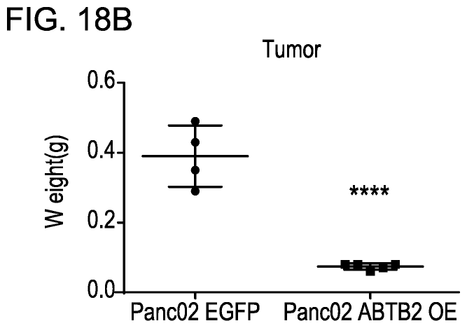

ABTB2 overexpression impact on orthotopic pancreatic tumor growth was investigated in immunodeficiency mice. Stable mouse Panco2 cells with ABTB2-overexpression and control cells were cultured and harvested, then injected into immunodeficiency mice by orthotopic inoculation at a dose of 0.25×10⁶ cells per mouse. 35 Days later, the mice were euthanized to harvest different organs and tumors. The tumors were imaged and weighed. Representative results show tumors and different organs (FIG. 18A). Accumulated results of tumor sizes (FIG. 18B).

As demonstrated in the Examples, Ankyrin-rich BTB/POZ domain-containing protein-2 (ABTB2) had a strong effect on suppressing PaC growth. Using established stable PaC cells with ABTB2 knockout or overexpression, ABTB2 had a critical role in PaC tumorigenesis and metastasis. Intrinsic overexpression of ABTB2 had a statistically significant effect on suppressing PaC tumor initiation and progression. An ABTB2-expressing virus under the control of Cox-2 promoter that ensures specific expression of ABTB2 in PaC tumors was constructed using the adeno-associated virus serotype 2 (AAV2), which has been widely used in PaC gene therapy due to its high affinity for PaC. The resultant therapeutic virus, named mAbtb2-AAV2, infects PaC cells and specifically expresses ABTB2 in PaC tumors. Treating PaC-bearing mice with this therapeutic virus showed a statistically significant effect on suppressing tumor growth with no detectable adverse effects. These results indicate that ABTB2-targeted therapy is a promising PaC treatment.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A vector comprising a nucleic acid encoding an Ankyrin-rich BTB/POZ domain-containing protein-2 (ABTB2).

2. The vector of claim 1, wherein the vector is selected from a viral vector and a non-viral vector.

3. The vector of claim 2, wherein the non-viral vector is a lentiviral vector.

4. The vector of claim 2, wherein the viral vector is an adeno-associated viral vector.

5. The vector of claim 4, wherein the viral vector is selected from the group consisting of an adeno-associated-virus serotype-1 (AVV-1) viral vector, an adeno-associated-virus serotype-2 (AVV-2) viral vector, adeno-associated-virus serotype-5 (AVV-5) viral vector, adeno-associated-virus serotype-6 (AVV-6) viral vector, adeno-associated-virus serotype-8 (AVV-8) viral vector, adeno-associated-virus serotype-9 (AVV-9) viral vector, adeno-associated-virus serotype-rh74 (AVV-rh74) viral vector, adeno-associated-virus-2i8 (AVV-2i8) viral vector, adeno-associated-virus-B1 (AVV-B1) viral vector, adeno-associated-virus-CAM130 (AVV-CAM130) viral vector, adeno-associated-virus-M41 (AVV-M41) viral vector, adeno-associated-virus MTP (AAV587MTP and AAV588MTP) viral vector, adeno-associated-virus NP22 (AAV-NP22) viral vector, adeno-associated-virus NP66 (AAV-NP66) viral vector, adeno-associated-virus MYO (AAVMYO) viral vector, an adeno-associated-virus tyrosine mutant viral vector, and an ancestral adeno-associated-virus (ancAVV) viral vector.

6. The vector of claim 1, further comprising a promoter.

7. The vector of claim 6, wherein the promoter is selected from the group consisting of a Cox-2 promoter, a P48 promoter, a Pdx1 promoter, a Hnf6 promoter, a ngn3 promoter, a MAFA promoter, a NeuroD/BETA2 promoter, a Pax6 promoter, and a Pax4 promoter.

8. A composition comprising a vector comprising a nucleic acid encoding an Ankyrin-rich BTB/POZ domain-containing protein-2 (ABTB2).

9. The composition of claim 8, wherein the vector is selected from a viral vector and a non-viral vector.

10. The composition of claim 8, wherein the viral vector is an adeno-associated viral vector.

11. The composition of claim 10, wherein adeno-associated viral vector.

12. The composition of claim 11, wherein the adeno-associated viral vector is selected from the group consisting of an adeno-associated-virus serotype-1 (AVV-1) viral vector, an adeno-associated-virus serotype-2 (AVV-2) viral vector, an adeno-associated-virus serotype-5 (AVV-5) viral vector, an adeno-associated-virus serotype-6 (AVV-6) viral vector, an adeno-associated-virus serotype-8 (AVV-8) viral vector, an adeno-associated-virus serotype-9 (AVV-9) viral vector, an adeno-associated-virus serotype-rh74 (AVV-rh74) viral vector, an adeno-associated-virus-2i8 (AVV-2i8) viral vector, an adeno-associated-virus-B1 (AVV-B1) viral vector, an adeno-associated-virus-CAM130 (AVV-CAM130) viral vector, an adeno-associated-virus-M41 (AVV-M41) viral vector, an adeno-associated-virus MTP (AAV587MTP and AAV588MTP) viral vector, an adeno-associated-virus NP22 (AAV-NP22) viral vector, adeno-associated-virus NP66 (AAV-NP66) viral vector, an adeno-associated-virus MYO (AAVMYO) viral vector, an adeno-associated-virus tyrosine mutant viral vector, and an ancestral adeno-associated-virus (ancAVV) viral vector.

12. The composition of claim 8, wherein the vector further comprises a promoter selected from the group consisting of a Cox-2 promoter, a P48 promoter, a Pdx1 promoter, a Hnf6 promoter, a ngn3 promoter, a MAFA promoter, a NeuroD/BETA2 promoter, a Pax6 promoter, and a Pax4 promoter.

13. The composition of claim wherein the composition is a pharmaceutical composition.

14. A method for treating cancer in a subject in need thereof, the method comprising: administering to the subject a composition comprises a vector comprising a nucleic acid encoding an Ankyrin-rich BTB/POZ domain-containing protein-2 (ABTB2).

15. The method of claim 14, wherein the subject in need thereof has or is suspected of having pancreatic cancer.

16. The method of claim 14, wherein the vector is an adeno-associated viral vector is selected from the group consisting of an adeno-associated-virus serotype-1 (AVV-1) viral vector, an adeno-associated-virus serotype-2 (AVV-2) viral vector, an adeno-associated-virus serotype-5 (AVV-5) viral vector, an adeno-associated-virus serotype-6 (AVV-6) viral vector, an adeno-associated-virus serotype-8 (AVV-8) viral vector, an adeno-associated-virus serotype-9 (AVV-9) viral vector, an adeno-associated-virus serotype-rh74 (AVV-rh74) viral vector, an adeno-associated-virus-2i8 (AVV-2i8) viral vector, an adeno-associated-virus-B1 (AVV-B1) viral vector, an adeno-associated-virus-CAM130 (AVV-CAM130) viral vector, an adeno-associated-virus-M41 (AVV-M41) viral vector, an adeno-associated-virus MTP (AAV587MTP and AAV588MTP) viral vector, an adeno-associated-virus NP22 (AAV-NP22) viral vector, adeno-associated-virus NP66 (AAV-NP66) viral vector, an adeno-associated-virus MYO (AAVMYO) viral vector, an adeno-associated-virus tyrosine mutant viral vector, and an ancestral adeno-associated-virus (ancAVV) viral vector.

17. The method of claim 14, wherein the vector further comprises a promoter selected from the group consisting of a Cox-2 promoter, a P48 promoter, a Pdx1 promoter, a Hnf6 promoter, a ngn3 promoter, a MAFA promoter, a NeuroD/BETA2 promoter, a Pax6 promoter, and a Pax4 promoter.

18. The method of claim 14, wherein pancreatic cancer cells express the nucleic acid encoding the ABTB2.

19. The method of claim 14, wherein the administration reduces pancreatic tumor growth.

20. The method of claim 14, wherein the administration is by intravenous injection.
